(19) United States
(12) Patent Application Publication
(10) Pub. No.: US 2016/0158480 A1
(43) Pub. Date: Jun. 9, 2016

(54) TUBE FOR BREATH-INDUCING AND APPARATUS FOR BREATH-INDUCING USING THE SAME

(71) Applicant: Korea University Research and Business Foundation, Seoul (KR)

(72) Inventors: Jaekwan LIM, Bucheon-si (KR); Seung-Zhoo YOON, Seoul (KR)

(73) Assignee: Korea University Research and Business Foundation, Seoul (KR)

(21) Appl. No.: 14/561,476

(22) Filed: Dec. 5, 2014

(51) Int. Cl. A61M 16/10; A61M 16/16; A61M 16/04; A61M 16/08; A61M 16/00; A61M 16/06

(52) U.S. Cl. CPC A61M 16/109; A61M 16/0003; A61M 16/06; A61M 16/04; A61M 16/0875; A61M 16/105; A61M 16/16; A61M 16/0057; A61M 2205/3368; A61M 2205/7536

(57) ABSTRACT

A breath-inducing tube to assist a patient's breathing and a breath-inducing apparatus using the same are disclosed. The breath-inducing tube can include: a divider having a connection port, an intake port and an exhaust port; a connection tube which is connected to the connection port such that the connection tube is connected to a patient; an exhaust tube which is connected to the exhaust port to discharge a patient's breathing gas; an inhalation part including a first inhalation tube of which one end is connected to the intake port and a second inhalation tube; and a heating module which has one end connected to the other end of the first inhalation tube and another end connected to the second inhalation tube to apply heat to the breathing gas introduced through the second inhalation tube and the first inhalation tube.

### TECHNICAL FIELD

The present invention relates to a breath-inducing tube and a breath-inducing apparatus using the same and in particular to a breath-inducing tube and a breath-inducing apparatus using the same which solve problems of the humidification associated with the inducement of a patient's breath and which has a simple structure and is able to maintain humidity and a temperature.

### BACKGROUND ART

For a patient who is unconscious under anesthesia during the operation or a patient who is unconscious in the intensive care unit, a breath-inducing apparatus such as an artificial breathing device is used to assist a patient's breathing for the artificial and forced inducement of breath.

Generally, an anesthetic or oxygen is supplied to a patient through an anesthetic apparatus or an artificial breathing device by means of a gas supply device which is embedded in the wall of an operating room or a sickroom. Then, a breath-inducing tube is mounted on the artificial breathing device to supply a breathing gas such as an anesthetic or oxygen through patient's respiratory organs.

In general, a breath-inducing tube consists of an inhalation tube for introducing a gas for breathing (hereinafter referred to as ‘breathing gas’) such as an anesthesia gas or oxygen such that a patient can inhale a breathing gas, an exhaust tube which branches off from the inhalation tube and discharges CO₂ or remaining anesthesia gas which is discharged after a patient's respiration, and a divider which branches off to be connected to an outlet of the inhalation tube and an inlet of the exhaust tube and which is connected to an exhauster provided to a patient's mouth.

An anesthetic provided to a patient is supplied in such a manner that a liquid-type volatile inhalation anesthetic is evaporated by a vaporizer and is supplied in the form of gas, and oxygen which is a gas for breathing is also supplied in the form of gas. An anesthetic gas or a breathing gas has a low temperature such that a patent feels the chill and thus has an adverse effect on a patient. Further, since an anesthetic gas or a breathing gas is dry without moisture, it may make a bronchus or a lung of a patient dry.

To overcome the problems associated with the moisture or temperature of breathing gas, techniques for heating and humidifying breathing gas which is provided to a patient are suggested. In one example, Korean patent No. 10-1143805 titled ‘evaporator system for heating and humidifying anesthetic gas and breathing gas’ discloses a technique which maintains adequate temperature and humidity during the introduction of breathing gas for the general anesthesia and the supply of oxygen.

FIG. 1 shows a vaporizer system disclosed in the above Korean Patent in which heat wires and a temperature and humidity sensor are mounted on a breath tube. As shown in FIG. 1, heat wires are arranged along the length of the inhalation tube 30 in the inhalation tube 30 to apply heat to a breathing gas which is introduced through the inhalation tube 30. Also, a technique is disclosed which vaporizes distilled water which is filled in a cartridge by ultrasound vibration and which controls humidity of a breathing gas.

Besides the humidification method disclosed in the above Korean patent, a method for maintaining humidity is suggested in which a piece of cloth is wrapped around heat wires embedded in the inhalation tube and in which water is evaporated from a wet cloth by heat generated from heat wires.

However, in case that humidified breathing gas is supplied through an inhalation tube as described in the above, water which is sprayed by the humidification is supposed to be condensed and be gathered in the inhalation tube or in the exhaust tube. As such, if gathered water is introduced into an anesthesia machine, a malfunction of a sensor of the anesthesia machine can be caused.

Further, the use of the breathing tube for long periods of time causes water in the gathered water or the breathing tube to create surroundings which generate bacilli or bacteria. This has an adverse effect on a patient and causes the breathing tube to be replaced frequently.

Moreover, to apply heat to a breathing gas, heat wires are installed along the inside of the inhalation tube as shown in FIG. 1. This makes the structure of the breathing tube complex and also the replacement of the entire heat wires or the replacement of the inhalation tube itself is required when heat wires are broken, thereby increasing the cost.

### DISCLOSURE OF THE INVENTION

### Technical Problem

To solve the above problems, the present invention provides a breath-inducing tube and a breath-inducing apparatus using the same which solve problems associated with the humidification by the inhalation tube and in which the structure of the breath-inducing tube is simple and an easy replacement and the maintenance of humidity and temperature are possible.

### Technical Solution

To achieve the object of the invention, the invention provides a breath-inducing tube for assisting a patient's breathing comprising: a divider having a connection port, an intake port and an exhaust port; a connection tube which is connected to the connection port of the divider such that the connection tube is connected to a patient; an exhaust tube which is connected to the exhaust port of the divider to discharge a patient's breathing gas; an inhalation part having a first inhalation tube of which one end is connected to the intake port of the divider and a second inhalation tube; and a heating module which has one end connected to the other end of the first inhalation tube and the other end connected to the second inhalation tube to make the first inhalation tube in fluid communication with the second inhalation tube and which applies heat to the breathing gas introduced through the second inhalation tube and the first inhalation tube.

Here, a plurality of the second inhalation tubes and a plurality of the heating modules are provided and the heating module and the second inhalation tube are connected in consecutive order.

Also, the first inhalation tube and the second inhalation tube are detachably connected to the heating module.

Also, the heating module comprises: a module body which has a hollow part formed along the direction of connecting the first inhalation tube and the second inhalation tube; the first connection part and the second connection part which are arranged on both sides of the module body in the direction of connecting the first inhalation tube and the second inhalation tube such that the first inhalation tube or the second inhalation tube is connected; and a heating member which is arranged in the hollow part of the module body to apply heat to the breathing gas.

Also, the heating module further comprises a power connector which supplies electric power from the outside to the heating member to apply heat to the heating member.

Also, the heating member has a filament shape to make the breathing gas flow through the hollow part of the module body.

Further, the breath-inducing tube further comprises a water blocking filter which is arranged between the connection tube and the divider to prevent water from being moved from the connection tube to the divider.

Meanwhile, the object of the present invention is achieved by a breath-inducing apparatus according to another embodiment of the present invention comprising: a breath-inducing tube; and a breath controller which is connected to the inhalation part of the breath-inducing tube and the exhaust tube and which induces a patient's breathing through the inhalation part and the exhaust tube.

Here, the breath-inducing apparatus further comprises a temperature sensor which is arranged on the divider of the breath-inducing tube to determine a temperature of the breathing gas introduced into the divider by the inhalation part of the breath-inducing tube, and wherein the breath controller controls the heating module such that the temperature of the breathing gas introduced into the divider is maintained at a predetermined temperature based on the measurement measured by the temperature sensor.

Also, the breath controller comprises a temperature display which displays temperature measured by the temperature sensor; and a temperature controller which controls heating temperature of the heating module by user's manipulation.

### Advantageous Effect

According to the invention, a breath-inducing tube and a breath-inducing apparatus using the same are provided which solve problems associated with the humidification by the inhalation tube and in which the structure of the breath-inducing tube is simple and an easy replacement and the maintenance of the humidity and the temperature are possible.

### BRIEF DESCRIPTION OF THE DRAWINGS

FIG. 1 represents a vaporizer disclosed in Korean Patent No. 10-1143805 in which heat wires and a temperature/humidity sensor are installed.

FIG. 2 represents a breath-inducing apparatus according to the present invention.

FIGS. 3 and 4 represent a breath-inducing apparatus according to the first embodiment of the present invention.

FIG. 5 represents an example of a heating module according to the present invention.

FIG. 6 represents a breath-inducing tube according to the second embodiment of the present invention.

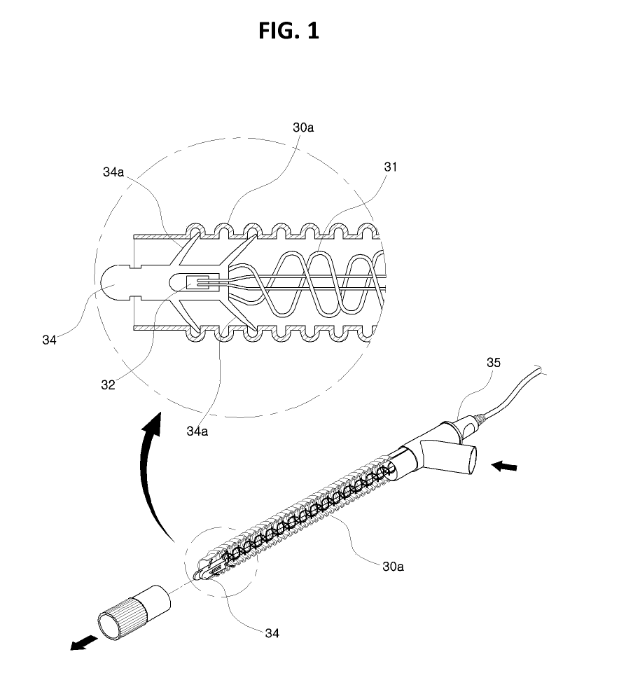

### BEST MODE FOR CARRYING OUT THE INVENTION

Hereinafter, preferred embodiments will be explained in detail referring to attached drawings. A breath-inducing tube 300 and a breath-inducing apparatus 1 using the same according to the present invention are applicable to put a patient under general anesthesia and provide a patient with oxygen during the operation and are also used to practice artificial respiration for a patient who cannot make a spontaneous respiration such as a serious case. Here, a breathing gas supplied by the breath-inducing tube 300 comprises an anesthesia gas and a gas for breathing such as oxygen which are referred to as ‘breathing gas’ hereinafter.

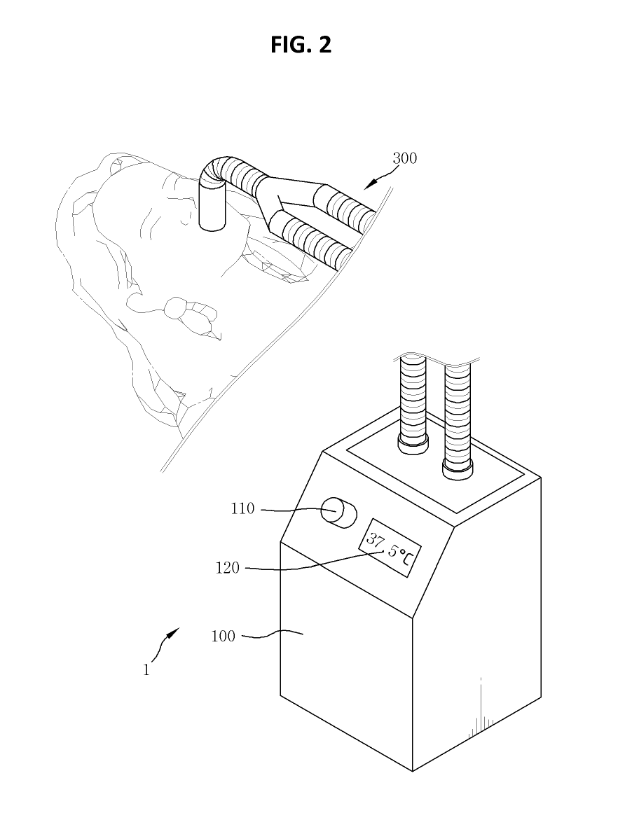

FIG. 2 shows a breath-inducing apparatus 1 according to the present invention. As shown in FIG. 2, the breath-inducing apparatus 1 comprises a breath-inducing tube 300 and a breath controller 100.

The breath-inducing tube 300 is arranged for a patient to assist the breathing of a patient under the anesthetic for the operation or the breathing of a patient who cannot make a spontaneous respiration in intensive care unit as shown in FIG. 2 and to induce a patient's respiration by a breathing gas from the breathing controller 100.

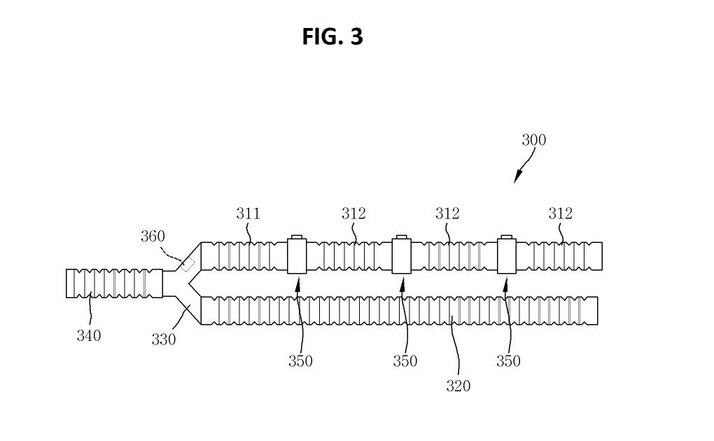

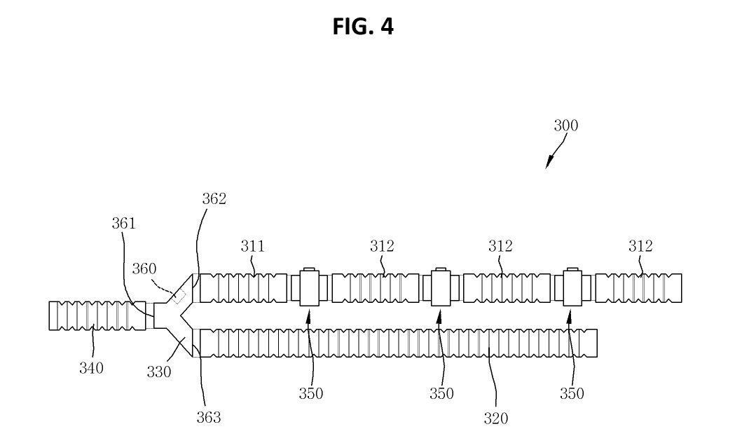

FIGS. 3 and 4 represent the arrangement of the breath-inducing tube 300 according to the first embodiment of the present invention. Referring to FIGS. 3 and 4, the breath-inducing tube 300 comprises a divider 330, a connection tube 340, an exhaust tube 320, inhalation part 311 and 312 and a heating module 350.

The divider 330 interconnects the connection tube 340, the inhalation part 311 and 312 and the exhaust tube 320 such that a breathing gas introduced by the inhalation part is supplied to a patient through the connection tube 340 and a breathing gas discharged from a patient through the connection tube 340, i.e., CO₂ or the remaining anesthetic gas, is discharged by the exhaust tube 320.

In one example of the present invention, the divider 330 comprises a Y-shaped arrangement having a connection port 331, an intake port 332 and an exhaust port 333. Also, there is no restriction to the shape as long as the connection tube 340, the inhalation part 311 and 312 and the exhaust tube 320 are interconnected and therefore a T-shaped arrangement is possible.

The connection tube 340 is connected to the connection port 331 of the divider 330 so that it is connected to a patient as shown in FIG. 4. That is, one end of the connection tube is connected to the connection port 331 of the divider and the other end is connected to a breathing device which is inserted into or installed in a patient's mouth or nose as shown in FIG. 2.

Here, the breathing device connected to the connection tube 340 can be provided in the form of a mask or a tube which is inserted into a patient's respiratory tract. Further, in one example of the present invention, the connection tube 340 and the breathing device are configured to be separated from each other. Alternatively, a mask or a respiratory-tract insertion tube can be connected to the connection port 331 of the divider 330. That is, the connection tube 340 of the present invention is defined to comprise a breathing device which is connectable to the connection port 331 of the divider 300.

One end of the exhaust tube 320 is connected to the exhaust port 333 of the divider 330 and the other end of the exhaust tube is connected to the breath controller 100 so that a patient's breathing gas is discharged through the connection tube 340 and the exhaust port 333 of the divider 330.

In one example, the exhaust tube 320 is configured to be separated from the divider 330 so that it is possible to sterilize or replace only the exhaust tube 320. Here, the exhaust tube 320 has bellows shape to be bent, compressed or elongated easily as shown in FIGS. 3 and 4.

One end of the inhalation part 311 and 312 is connected to the intake 332 and the other is connected to the breath controller 100 to supply breath gas to a patient by the divider 330 and the connection tube 340. Here, the inhalation part 311 and 312 may comprise the first inhalation tube 311 and the second inhalation tube 312.

In the present invention, a plurality of the second inhalation tubes 312 of the inhalation part 311 and 312 and a plurality of the heating modules 350 are provided as shown in FIGS. 3 and 4. In particular, three heating modules 350 and three second inhalation tubes 312 are provided in FIGS. 3 and 4.

One end of the first inhalation tube 311 is connected to the intake port 332 of the divider 330 and the other end is connected to the heating module 350. The heating module 350 and the second inhalation tube 312 are connected in consecutive order and the heating module 350 connected to the other end of the first inhalation tube 311 will be the first heating module.

In more detail, referring to FIG. 4, one end of the first inhalation tube 311 is connected to the intake port 332 of the divider 330 and the other end is connected to the first connection part 352 of the heating module 350 which will be described below. Then, one end of the first one of the second inhalation tubes 312 is connected to the second connection part 353 of the heating module 350 and the other end of the first one of the second inhalation tubes 312 is connected to the first connection part 352 of the second heating module 350.

Similarly, one end of the second one of the second inhalation tubes 312 is connected to the second connection part 353 of the second heating module 350 and the other end of the second one of the second inhalation tubes 312 is connected to the first connection part 352 of the third heating module 350. Then, one end of the third one of the second inhalation tubes 312 is connected to the second connection part 353 of the third heating module 350 and the other end of the third one of the second inhalation tubes 312 is connected to the breath controller 100 so that the connection of the inhalation part 311 and 312 for the intake of a breathing gas is accomplished.

Meanwhile, the heating module 350 applies heat to breathing gas introduced by the inhalation part 311 and 312, i.e., the first inhalation tube 311 and the second inhalation tube 312, to maintain the temperature of the breathing gas introduced by the inhalation part 311 and 312.

Here, the first inhalation tube 311 and the second inhalation tube 312 may be detachably connected to the heating module 350. Therefore, when the heating module 350 is required to be replaced due to the trouble, it is possible to replace the corresponding heating module 350 only. Similarly, when the first connection tube 340 and the second connection tube 340 are required to be replaced due to the contamination or damage, it is possible to replace the corresponding part only.

Further, it is possible to increase or decrease the number of heating sites on the inhalation part 311 and 312 by varying the number of the heating module 350 as needed. It is also possible to vary the length of the inhalation part 311 and 312 by increasing the number of the heating module 350 and the second inhalation tubes 312 or by increasing the length of the second inhalation tube 312.

Since the heating module 350 for heating an intake gas is not installed inside the first inhalation tube 311 or the second inhalation tube 312, the structure of the inhalation part 311 and 312 is simple and when any of the heating modules 350 is out of order, it is possible to replace the corresponding heating module 350 only without need to alter the first inhalation tube 311, the second inhalation tube 312 and the other heating module 350.

Here, the connection tube 340 of the breath-inducing tube 300 and the divider 330, the first inhalation tube 311 and the divider 330, and the discharge tube 320 and the divider 330 are all detachably provided to facilitate the sterilization or the replacement.

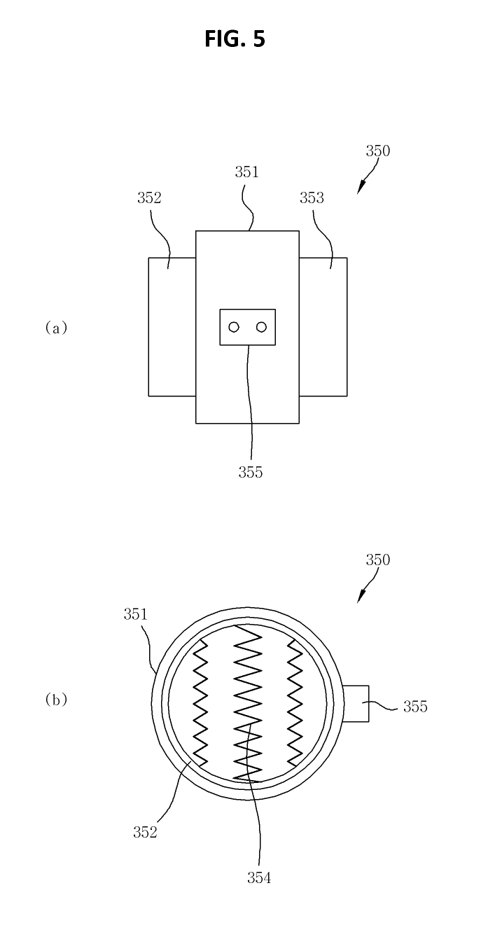

Meanwhile, FIG. 5 represents the heating module 350 according to the present invention. FIG. 5(a) is a side view of the heating module 350 and FIG. 5(b) is a front view of the heating module 350.

Referring to FIG. 5, the heating module 350 according to the present invention comprises a module body 351, the first connection part 352, the second connection part 353 and a heating member 354.

The module body 351 is in the shape of a tube which is hollow in the direction of connecting the first inhalation tube 311 and the second inhalation tube 312. The first connection part 352 and the second connection part 353 are arranged on both sides of the module body 351 in the direction of connecting the first inhalation tube 311 and the second inhalation tube 312.

Here, the first inhalation tube 311 or the second inhalation tube 312 is connected to the first connection part 352 and the second connection part 353 so that the heating module 350 is arranged between the first inhalation tube 311 and the second inhalation tube 312 or between the second inhalation tubes 312.

The heating member 354 is installed in the hollow part of the module body 351 to heat a breathing gas. The heating member 354 according to the present invention is arranged in the form of a heat wire which is heated by the electric power supplied from the outside and is arranged in the form of filaments to enable the gas to flow through the hollow part of the module body 351 as shown in FIG. 5(b).

Here, the heating module 350 comprises a power connector 355 which supplies power from the outside to the heating member 354 to heat the heating member 354 and the connection of a power cable to the power connector 355 makes it possible to supply power from the outside.

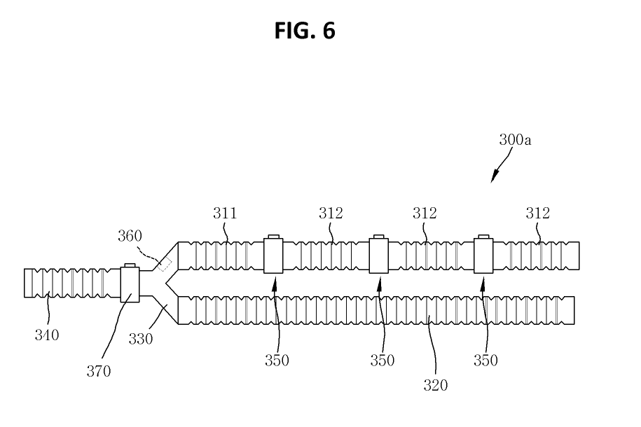

Hereinafter, referring to FIG. 6, a breath-inducing tube 300a according to the second embodiment of the present invention will be described. When the breath-inducing tube 300a according to the second embodiment of the present invention is described, the same reference numerals will be used to indicate the corresponding elements of the breath-inducing tube 300 according to the first embodiment and the descriptions of the elements corresponding to the first embodiment may be omitted.

Referring to FIG. 6, the breath-inducing tube 300a according to the second embodiment of the present invention comprises a water blocking filter 370. The water blocking filter 370 is arranged between the connection tube 340 and the divider 330 to block the movement of water from the connection tube 340 to the divider 330.

According to the above arrangement, water discharged from a patient's body during breathing is prevented from being moved to the divider 330 by the water blocking filter 370 and therefore stays in the connection tube 340. Then, a breathing gas is heated by the heating module 350 during breathing and then is introduced through the divider 330. Then the breathing gas is humidified by water in the connection tube 340 and is introduced to the patient's body such that the heated and humidified breathing gas is introduced into the patient's body.

Further, water generated during patient's breathing is prevented from being moved to the exhaust tube 320 or from being introduced into the inhalation part 311 and 312 or into anesthetic gas and therefore contamination problems caused by the prior humidification system can be solved out.

Meanwhile, the breath-inducing apparatus 1 according to the present invention may comprise a temperature sensor 360 as shown in FIGS. 3, 4 and 6. The temperature sensor 360 is arranged in the divider 330 of the breath-inducing tube 300 and determines the temperature of a breathing gas introduced into the divider 330 by the inhalation part 311 and 312 of the breath-inducing tube 300.

Here, the breath controller 100 controls the heating module 350 based on the measurements of the temperature sensor 360 such that the temperature of a breathing gas introduced into the divider 330 is maintained at a predetermined temperature. Therefore, the temperature of a breathing gas introduced into the patient can be maintained on a certain level.

Further, the breath controller 100 comprises a temperature display 120 and a temperature controller 110 as shown in FIG. 2. The temperature display 120 displays the temperature measured by the temperature sensor 360 so that a doctor or a nurse can visually check the temperature of a breathing gas introduced into the patient and monitor the current status.

Also, the control of the heating temperature of the heating module 350 by the manipulation of the temperature controller 110 may lead to the control of the temperature of the breathing gas introduced into the patient based on the patient's status.

In the embodiments described in the above, the breath-inducing tube 300 consists of three heating modules 350 and three second inhalation tubes 312 as shown in FIGS. 3, 4 and 6. However, it should be appreciated that the breath-inducing tube may consist of one heating module 350 and one second inhalation tube 312, two heating modules 350 and two second inhalation tubes 312, or four or more heating modules 350 and four or more second inhalation tubes 312.

It is intended that the foregoing description has described only a few of the many possible implementations of the present invention, and that variations or modifications of the embodiments apparent to those skilled in the art are embraced within the scope and spirit of the invention. The scope of the invention is determined by the claims and their equivalents.

LIST OF REFERENCE NUMERALS

| 1: breath-inducing apparatus | 100: breath controller |
|---|---|
| 110: temperature controller | 120: temperature display |
| 300, 300a: breath-inducing tube | |
| 311: a first inhalation tube | 312: a second inhalation tube |
| 320: exhaust tube | 330: divider |
| 331: connection port | 332: intake port |
| 333: exhaust port | 340: connection tube |
| 350: heating module | 351: module body |
| 352: a first connection part | 353: a second connection part |
| 354: heating member | 355: power connector |
| 360: temperature sensor | 370: water blocking filter |

1. A breath-inducing tube for assisting a patient's breathing comprising:
a divider having a connection port, an intake port and an exhaust port;
a connection tube which is connected to the connection port of the divider such that the connection tube is connected to a patient;
an exhaust tube which is connected to the exhaust port of the divider to discharge a patient's breathing gas;
an inhalation part having a first inhalation tube of which one end is connected to the intake port of the divider and a second inhalation tube; and
a heating module which has one end connected to the other end of the first inhalation tube and the other end connected to the second inhalation tube to make the first inhalation tube in fluid communication with the second inhalation tube and which applies heat to the breathing gas introduced through the second inhalation tube and the first inhalation tube.

2. The breath-inducing tube according to claim 1, wherein a plurality of the second inhalation tubes and a plurality of the heating modules are provided and the heating module and the second inhalation tube are connected in consecutive order.

3. The breath-inducing tube according to claim 2, wherein the first inhalation tube and the second inhalation tube are detachably connected to the heating module.

4. The breath-inducing tube according to claim 2, wherein the heating module comprises:
a module body which has a hollow part formed along the direction of connecting the first inhalation tube and the second inhalation tube;
a first connection part and a second connection part which are arranged on both sides of the module body in the direction of connecting the first inhalation tube and the second inhalation tube such that the first inhalation tube or the second inhalation tube is connected; and
a heating member which is arranged in the hollow part of the module body to apply heat to the breathing gas.

5. The breath-inducing tube according to claim 4, wherein the heating module further comprises a power connector which supplies electric power from the outside to the heating member to apply heat to the heating member.

6. The breath-inducing tube according to claim 5, wherein the heating member has a filament shape to make the breathing gas flow through the hollow part of the module body.

7. The breath-inducing tube according to claim 1, further comprising a water blocking filter which is arranged between the connection tube and the divider to prevent water from being moved from the connection tube to the divider.

8. A breath-inducing apparatus for assisting a patient's breathing comprising:
a breath-inducing tube according to claim 1; and
a breath controller which is connected to the inhalation part of the breath-inducing tube and the exhaust tube and which induces a patient's breathing through the inhalation part and the exhaust tube.

9. The breath-inducing apparatus according to claim 8, further comprising a temperature sensor which is arranged on the divider of the breath-inducing tube to determine a temperature of the breathing gas introduced into the divider by the inhalation part of the breath-inducing tube, and wherein the breath controller controls the heating module such that the temperature of the breathing gas introduced into the divider is maintained at a predetermined temperature based on the measurement measured by the temperature sensor.

10. The breath-inducing apparatus according to claim 9, wherein the breath controller comprises a temperature display which displays temperature measured by the temperature sensor; and a temperature controller which controls heating temperature of the heating module by user's manipulation.
